(19) United States
(12) Patent Application Publication
(10) Pub. No.: US 2022/0346848 A1
(43) Pub. Date: Nov. 3, 2022

(54) BONE SCREW

(71) Applicants: Lutz BIEDERMANN, VS-Villingen (DE); Timo BIEDERMANN, Trossingen (DE); Berthold DANNECKER, St. Georgen (DE)

(72) Inventors: Lutz BIEDERMANN, VS-Villingen (DE); Timo BIEDERMANN, Trossingen (DE); Berthold DANNECKER, St. Georgen (DE)

(21) Appl. No.: 17/731,088

(22) Filed: Apr. 27, 2022

Related U.S. Application Data
(60) Provisional application No. 63/182,250, filed on Apr. 30, 2021.

(30) Foreign Application Priority Data
Apr. 30, 2021 (EP) 21 171 669.1

(51) Int. Cl. A61B 17/86; A61B 17/70

(52) U.S. Cl. CPC A61B 17/863; A61B 17/7032; A61B 17/8635; A61B 17/704

(57) ABSTRACT

A bone screw includes a shank having a longitudinal axis, a tip configured to be inserted first into bone, a core, and a thread with a plurality of turns winding in a helix around at least part of the core to engage bone. The thread has a lower flank and an upper flank, wherein for a first section of the thread, the upper and lower flanks form a first angle for at least one full turn, and wherein for a second section of the thread, the upper and lower flanks form a second angle for at least one full turn that is greater than the first angle. An outer diameter of the second section adjacent to the first section is at least as wide as an outer diameter of the first section adjacent to the second section.

### CROSS-REFERENCE TO RELATED APPLICATION(S)

The present application claims priority to and the benefit of U.S. Provisional Patent Application No. 63/182,250, filed Apr. 30, 2021, the contents of which are hereby incorporated by reference in their entirety, and claims priority from European Patent Application EP 21 171 669.1, filed Apr. 30, 2021, the contents of which are hereby incorporated by reference in their entirety.

### BACKGROUND

### Field

The application relates to a bone screw. In particular, the application relates to a bone screw that may be used in orthopedic surgery, and more particularly, in spinal surgery.

### Description of Related Art

In orthopedic surgery, bone screws that have a thread on their outer surface configured to engage bone are widely used for anchoring stabilization devices such as rods and/or plates to the bone. Various developments of bone screws have been made to fulfil specific requirements, such as a self-cutting ability and/or an increased resistance to pull-out forces.

U.S. Pat. No. 5,120,171, for example, describes a bone screw having a helical thread that gradually increases in thickness from the tip to the head of the screw. The thin thread at the tip of the screw can be inserted into a bone with minimal tearing or cracking of the bone. The thicker threads ascending from the tip to the head of the screw displace bone against the superior (top) thread surfaces. This displacement of bone increases the screw's resistance to being pulled out of the bone.

### SUMMARY

It is an object of the invention to provide an improved bone screw and a method of manufacturing the bone screw which are simple and effective.

A bone screw according to an aspect of the invention includes a shank configured to be anchored in bone, wherein the shank has a screw axis defining a longitudinal direction, and further has a tip configured to be inserted first into bone. The shank includes a core and a thread that winds in a helix with a plurality of turns around at least a portion of the core, the thread being configured to engage bone and having an upper flank facing away from the tip and a lower flank facing towards the tip. In a first section of the thread that is closer to the tip, the upper flank and the lower flank form a first angle with each other and in a second section of the thread that is farther away from the tip than the first section, the upper flank and the lower flank form a second angle with each other that is greater than the first angle.

Due to the smaller thread angle in the first section at or close to the tip of the bone screw, the thread is more sharp in the first section, which allows engagement of the cortical bone more easily. This may facilitate easier insertion of the bone screw into bone. The necessary insertion torque may be reduced compared to a thread which has a greater angle between the upper flank and the lower flank in the region at or adjacent to the tip.

Moreover, due to the greater thread angle in the second section that is closer to the head of the bone screw, resistance against pull-out forces, which tend to pull the bone screw out of the bone once it has been inserted, may be increased.

A method for manufacturing the bone screw includes a step of providing a bone screw with a bone thread having a constant thread angle along a substantial part of the length of the thread, and a step in which, in a region that extends from a position at or close to the tip along a portion of the shank, a thread with a smaller thread angle is cut, for example, milled. Hence, the method is easy to perform and cost saving.

### BRIEF DESCRIPTION OF THE DRAWINGS

Further features and advantages of the invention will become apparent from the description of embodiments by means of the accompanying drawings. In the drawings:

FIG. 1 shows a perspective exploded view of an embodiment of a polyaxial bone anchoring device, including a first embodiment of a bone screw.

FIG. 2 shows a perspective view of the polyaxial bone anchoring device of FIG. 1 in an assembled state.

FIG. 3 shows a cross-sectional view of the polyaxial bone anchoring device of FIGS. 1 and 2, the cross-section being taken in a plane including a screw axis of the bone screw and extending through a center of a receiving part of the polyaxial bone anchoring device perpendicular to a rod channel of the receiving part.

FIG. 4 shows an enlarged perspective view of a portion of a shank of the bone screw of FIGS. 1 to 3.

FIG. 5 shows a side view of the bone screw shown in FIGS. 1 to 4.

FIG. 6 shows a side view of the bone screw of FIG. 5 rotated by 90°.

FIG. 7 shows a cross-sectional view of the bone screw of FIGS. 5 and 6, the cross-section being taken in a plane extending through the center of the bone screw in the longitudinal direction.

FIG. 8 shows a side view of a second embodiment of a bone screw.

FIG. 9 shows a side view of the bone screw of FIG. 8 rotated by 90°.

FIG. 10 shows a side view of a third embodiment of a bone screw.

FIG. 11 shows a side view of the bone screw of FIG. 10 rotated by 90°.

### DETAILED DESCRIPTION

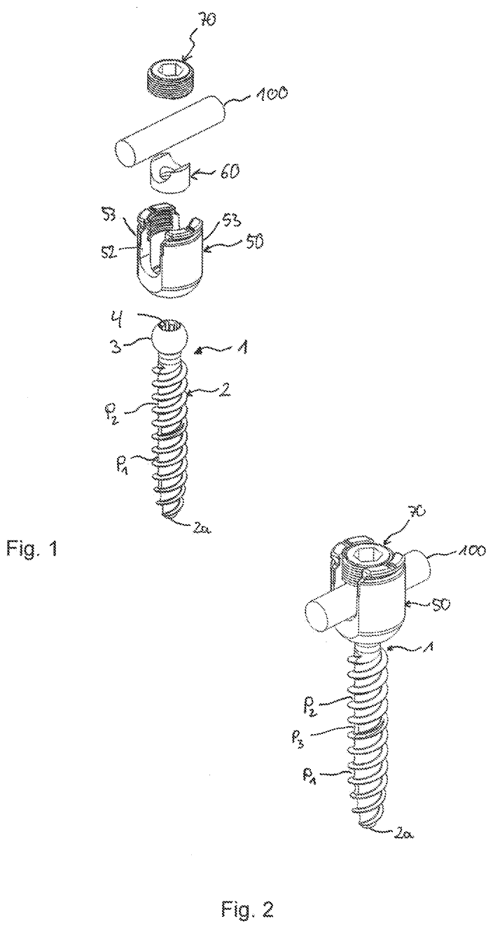

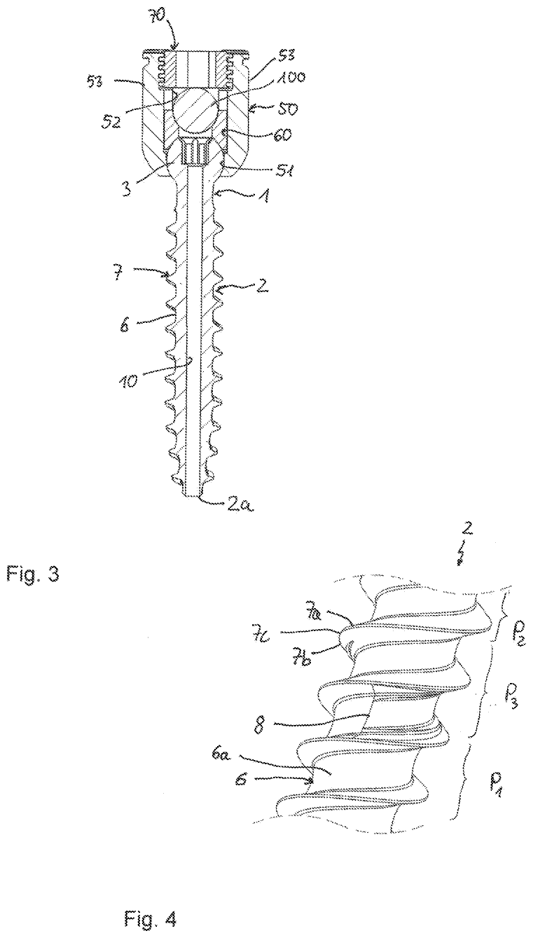

Referring to FIGS. 1 to 7, a first embodiment of a bone screw in connection with a polyaxial bone anchoring device according to an embodiment of the invention is described. The bone screw 1 includes a shank 2 with a tip 2a at one free end that is configured to enter the bone first. The tip may be blunt or sharp, or may have any suitable shape. At an end of the shank 2 opposite to the tip, a head 3 may be formed that may have a spherical outer surface portion and a free end surface 3a. More specifically, the head 3 may have the shape of a spherical segment, with a substantially flat end surface 3a. Further, an engagement recess 4 may be provided in the free end surface 3a of the head 3 for engagement with a drive tool. Adjacent to the head 3, a reduced diameter neck section 5 may be formed. The bone screw may be part of a polyaxial bone anchoring device. Such a polyaxial bone anchoring device typically includes the bone screw 1, a receiving part 50, and a pressure member 60. The receiving part 50 has an accommodation space with a seat 51 for the head 3 that enables the head 3 to pivot in the seat, similar to a ball and socket joint, so that the shank 2 can assume various angular positions relative to the receiving part. Furthermore, the receiving part 50 may have a recess 52 for receiving a spinal rod 100 and a locking member 70, for example, in the form of a screw member configured to cooperate with internally threaded legs 53 of the receiving part for locking the head 3 and the rod 100 in the receiving part 50. The head 3 can be locked at a particular angular position by tightening the locking member 70, which in turn exerts pressure via the rod and/or the pressure member 60 on the head 3. There may also be various different designs of such a polyaxial bone anchoring device, and the present disclosure is not limited to the design shown in the figures.

The shank 2 of the bone screw 1 has a core 6 and a thread 7 winding in a helix around the core 6 in a plurality of turns. In the specific embodiment, the thread 7 is a double start thread, i.e., two helices 7₁, 7₂ each having a separate thread entry wind around the core 6. By the thread 7, a longitudinal axis or screw axis S is defined, which also defines an axial direction of the bone screw 1. An outer diameter of the core or core diameter d, wherein d/2 is indicated in FIG. 7, may be constant over at least most of the length of the core 6. In a region adjacent to the tip 2a, the core diameter d may, however, decrease to form a tapered tip 2a. In the embodiment, the thread 7 extends from the tip 2a up to the neck section 5. In greater detail, the thread 7 includes a first or upper flank 7a facing towards the head 3, and a second or lower flank 7b facing towards the tip 2a. The upper flank 7a and the lower flank 7b join at a crest 7c at a radially outermost portion of the thread turn. The shape of the crest may be sharp or rounded or flat. A cross-section of the thread 7 in a plane including the screw axis S may be substantially triangular, in particular, with equilateral sides, and more specifically, the cross-section may be that of a V-thread. Moreover, a thread pitch, which is the distance in an axial direction from one crest 7c to a neighboring crest 7c, may be constant along substantially the entire length of the thread. In the case of the bone thread, the thread pitch may be such that there is a gap 6a between the thread turns on the core 6. The outer diameter D of the thread, which is twice the distance from the screw axis to the crest 7c, shown as D/2 in FIG. 7, may be constant along substantially the length of the thread. However, the outer diameter D may decrease in a region towards the tip 2a and/or in a run-out region towards the neck 5. As the thread 7 in this embodiment is a double start thread, a lead, i.e., a distance the screw travels with one revolution, is two times the pitch.

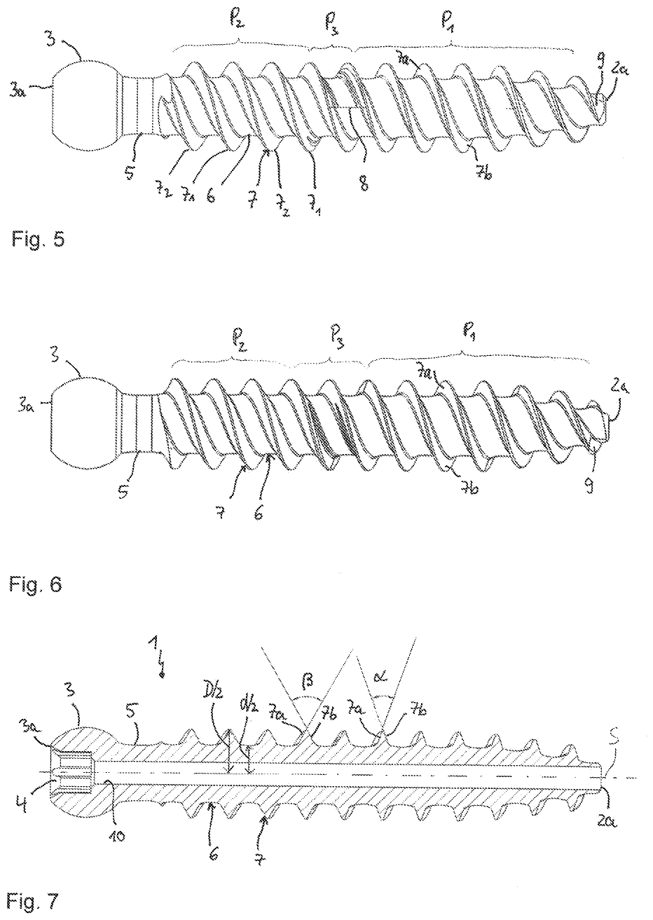

As best seen in FIGS. 5 to 7, in a first section P₁ of the thread 7 that starts at or close to the tip 2a, the upper flank 7a and the lower flank 7b of the thread 7 form a first angle α with each other. In a second section P₂ which is adjacent to or close to the neck section 5, the upper flank 7a and the lower flank 7b of the thread form a second angle β with each other. The first angle α is smaller than the second angle β, and as a result, the thickness of the thread in the axial direction is smaller in the first section than in the second section. The second angle β is in the embodiment 60°, which corresponds to the angle of a 60° V-thread. The first angle α is in the embodiment 40°. It should be noted that values for the first angle and the second angle for all embodiments cover at least manufacturing and/or measuring tolerances. The thread angle is measured between two flanks, more specifically the upper flank 7a and the lower flank 7b, in a plane including the screw axis S. In FIG. 7, the thread angle is shown as the angle formed by two intersecting planes extending through the upper flank 7a and the lower flank 7b, respectively, which generally also corresponds to the angle measured between two opposing flanks.

Preferably, the ratio between the first angle α and the second angle β is in a range between about 0.1 to 0.9, more preferably between 0.2 and 0.8, and still more preferably between 0.4 and 0.7.

Between the first section P₁ and the second section P₂, a transition section P₃ may be formed in which the thread angle changes between the first angle α and the second angle β. The transition section P₃ may be defined by a run-out zone where the tool that produces the thread with the first angle α runs out. Hence, along a portion of one turn of the thread in the transition zone P₃, the upper flank 7a and the lower flank 7b each have a portion with both the first thread angle α and also the second thread angle β. The transition section P₃ starts in the helical direction at a position 8 which corresponds to the start of the removal of a tool that generates the thread with the first angle α in the first section P₁. The transition section ends at an axial position in a direction towards the neck section 5, where the upper and lower flanks 7a, 7b are free from portions with the first thread angle α.

For facilitating insertion in the bone, the thread may have a cutting feature 9 at or close to the tip 2a. The cutting feature may be a recess in the thread that sharpens the thread, preferably in the first thread turn adjacent to the tip 2a. Moreover, the shank 2 may be conical in a region adjacent to the tip 2a. This can be achieved by either reducing the core diameter and/or the outer diameter of the thread 7.

Lastly, a channel 10 may extend from the end surface of the head 3 entirely through the shank 2 up to the tip 2a. This may be useful for guiding a guidewire therethrough or for injecting substances. The channel 10 is preferably coaxial with the shank axis S and has a circular cross-section. An inner diameter of the channel may be substantially constant over its length. However, the inner diameter may also vary along its length and/or the cross-section may have a shape other than circular. In a further embodiment (not shown), the shank may be fenestrated, i.e., the shank may have one or a plurality of openings that connect the channel 10 with the outside of the bone anchor.

A method of manufacturing the bone screw 1 includes at least a first step of providing a bone screw with a thread 7 along a length of the shank 2 that includes a portion at or close to the tip 2a, wherein the thread 7 has a thread angle that is constant along the length of the thread 7 and that corresponds to the second angle β in the second section P₂. The thread may be a V-thread and the second angle β may be 60°. Then, in a second step, a first section P₁ at or close to the tip 2a is generated that has the first thread angle α. The first angle α is smaller than the second angle β and may be, for example, 40°. Preferably, the first section P₁ is generated by machining the shank 2 with a tool, and more preferably, the thread is cut or milled with a thread cutter that starts at the tip and runs out from the position 8, which represents the beginning of a region where the tool is removed. The remaining section P₂ adjacent to the first section P₁, and more specifically adjacent to the transition section P₃, has the second angle β.

Generally, with this or a similar method, various other embodiments can be generated in which a first section is different from a second section with regard to the thread angle and/or with regard to the thread form. For example, the first section which is generated in the second step may be different from the second section also with regard to the threadform. Methods in which material is removed from a bone screw may be preferred as they are simple and cost saving. However, it can be also envisaged to generate the entire bone screw in one step using an additive manufacturing method, for example an additive layer manufacturing method such as laser or electron beam melting in which the bone screw is built up in a layer-wise manner based on CAD data of the final bone screw.

The bone anchor 1 may be made of any bio-compatible material, preferably however of titanium or stainless steel, or of any other bio-compatible metal or metal alloy or plastic material. For a bio-compatible alloy, a NiTi alloy, for example, Nitinol, may be used. Other materials that can also be used are magnesium or magnesium alloys. Bio-compatible plastic materials that can be used may be, for example, polyether ether ketone (PEEK) or poly-L-lactide acid (PLLA).

In clinical use, the bone screw is inserted into bone, for example, in the pedicle of a vertebra. In one method of use, the bone screw is first inserted into bone, and another device, such as a receiving part of a polyaxial bone anchoring device, which is exemplary shown in FIGS. 1 to 3, or a bone plate, is mounted onto the head 3 of the inserted bone screw. In another method of use, the bone screw is pre-assembled with the other device, and thereafter inserted into bone. When the bone screw is inserted, the first section P₁ of the shank 2 with the smaller first thread angle α facilitates the engagement of the thread 7 with the bone. In addition, the insertion torque is reduced. Thus, the bone screw can easily engage and traverse the cortical bone. Upon further insertion, the second section P₂ engages the bone until the bone screw is fully inserted. Due to the second section P₂ having the greater thread angle β, the resistance against pull-out is similar to that of a usual bone screw with a 60° V-thread extending along its entire length. In some instances, the resistance against pull-out may also be increased compared to a screw with a constant 60° V-thread, since the bone is condensed around the second section P₂ during insertion.

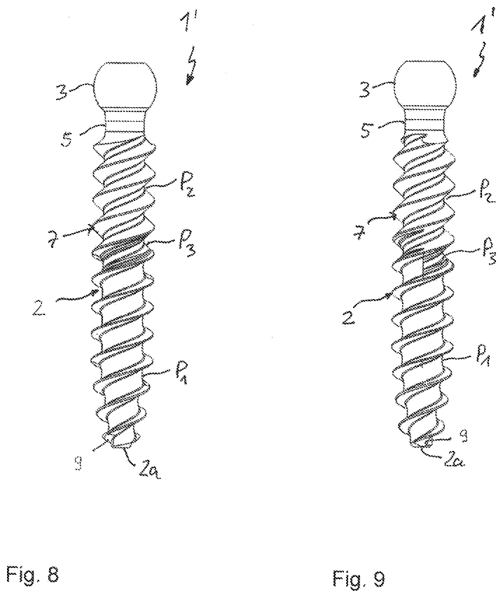

In FIGS. 8 and 9, a second embodiment of the bone screw is shown. The bone screw 1′ differs from the bone screw shown in FIGS. 1 to 7 only by the thread angles of the first and second sections. The second thread angle in the second section P₂ is in this case about 80°, and the first thread angle in the first section P₁ is about 40°. The second angle in this embodiment is about twice the first angle. Such a difference may be useful in the case of known or expected higher pull-out forces that may act on the bone screw.

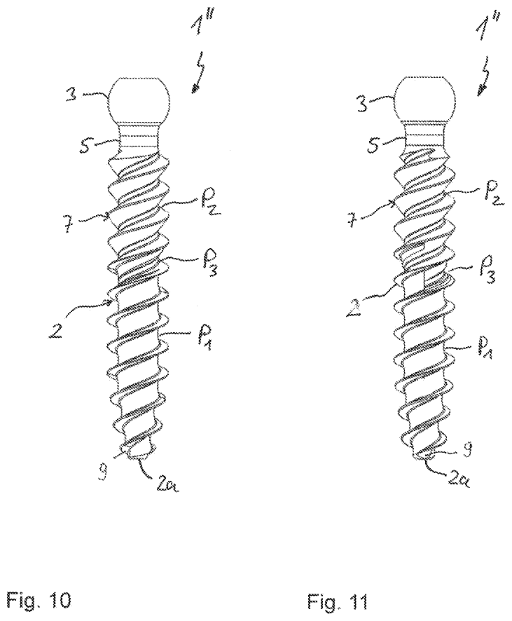

In FIGS. 10 and 11, a third embodiment of the bone screw is shown. The bone screw 1″ differs from the bone screw of the previous embodiments only by the thread angle. The second thread angle in the second section P₂ is about 50° and the first angle in the first section P₁ is about 30°. This configuration may be useful, for example, in the case of healthy bone for which a reduced insertion torque facilitates the insertion, while a risk of pull-out may not be overly high.

In the embodiments shown, the thread 7 is a double start thread. In a still further embodiment, the thread may be a single start thread or a triple or a multiple start thread. Also, in such embodiments, the bone screw may have first and second sections with corresponding thread angles as in the previous embodiments.

While the thread 7 is shown as a V-thread, other known threadforms can also be used as long as they fulfil the requirement of engaging bone. For example, trapezoidal threads, buttress threads, or any other threads may be used.

The specific thread shape, the pitch, the number of threads, etc., are parameters that may depend on the type of bone which the anchor is to be inserted into and on the purpose of the bone anchor.

While only two sections with different thread angles are described, it may be conceivable that more than two sections with different thread angles are formed. For example, a first section with a first thread angle could be formed adjacent to the tip, followed by a second section with a greater thread angle than that of the first section, and further followed by a third section with a greater thread angle than that of the second section in a direction towards the neck.

It shall further be noted that, while the pitch and the cross-section of the thread 7 remains substantially the same in the embodiments shown, there may be other embodiments where the pitch is different in the two sections having the different thread angles.

Other modifications may also be possible. For example, the shank may also have thread-free portions, i.e., the thread 7 may be present only on a portion or portions of the shank. The tip 2a does not need to be conical, but may be formed by the end surface of a cylindrical shank, or the end portion of the shank can have any other shape that is configured to be inserted into bone first. The bone screw may be headless or the head may have another shape. Various self-cutting features may also be provided, in particular in the first section, and/or the thread may be completely self-cutting.

For the polyaxial bone anchoring device, all kinds of polyaxial bone anchoring devices may be used.

While the present invention has been described in connection with certain exemplary embodiments, it is to be understood that the invention is not limited to the disclosed embodiments, but is instead intended to cover various modifications and equivalent arrangements included within the spirit and scope of the appended claims, and equivalents thereof.

What is claimed is:

1. A bone screw comprising:
a shank configured to be anchored in bone, the shank having a longitudinal axis and comprising a tip configured to be inserted first into bone, a core, and a thread with a plurality of turns winding in a helix around at least a portion of the core to engage bone;
a threadless neck at an end of the shank opposite the tip; and
a head connected to the shank by the neck and having a width measured perpendicular to the longitudinal axis that is greater than a maximum width of the neck;
wherein the thread comprises a lower flank that faces the tip and an upper flank that faces away from the tip, wherein for a first section of the thread, the upper flank and the lower flank form a first angle with each other for at least one full turn, and wherein for a second section of the thread located between the first section and the neck, the upper flank and the lower flank form a second angle with each other for at least one full turn that is greater than the first angle; and
wherein an outer diameter of every portion of the second section of the thread adjacent to the first section of the thread is at least as wide as an outer diameter of the first section of the thread adjacent to the second section of the thread.

2. The bone screw of claim 1, wherein the first and second sections of the thread are directly connected to each other.

3. The bone screw of claim 1, wherein respective pitches of the thread are the same in the first section and in the second section.

4. The bone screw of claim 1, wherein an outer diameter of the core is substantially constant along at least part of the first and second sections of the thread, including a transition region between the first and second sections.

5. The bone screw of claim 1, wherein the outer diameter of the thread is substantially constant along at least part of the first and second sections of the thread, including a transition region between the first section and second sections.

6. The bone screw of claim 1, wherein the first section of the thread comprises at least two turns.

7. The bone screw of claim 1, wherein the second section of the thread comprises at least two turns.

8. The bone screw of claim 1, wherein a length of the first section of the thread is at least a length of the second section of the thread in the longitudinal direction.

9. The bone screw of claim 1, wherein a ratio between the first angle and the second angle is between 0.4 and 0.7.

10. The bone screw of claim 1, wherein the first angle is between 20° and 50° and the second angle is between 30° and 80°.

11. The bone screw of claim 1, wherein the first section of the thread is adjacent to the tip.

12. The bone screw of claim 1, wherein a cross-section of the thread is symmetrical with respect to a plane perpendicular the longitudinal axis.

13. The bone screw of claim 1, further comprising a transition region between the first and second sections of the thread where at least one of the upper flank or the lower flank has a run-out portion in a direction of the helix.

14. The bone screw of claim 1, wherein the head has a spherical outer surface portion.

15. A polyaxial bone anchoring device comprising the bone screw of claim 14 and a receiving part for coupling the bone screw to a rod, the receiving part comprising a seat for pivotably receiving the head and a recess for receiving the rod.

16. The bone screw of claim 1, wherein the thread is devoid of any undercuts.

17. The bone screw of claim 1, wherein the first and second sections of the thread include a same number of helices as one another.

18. A method of manufacturing a bone screw, comprising:
providing a shank comprising a tip and a thread configured to engage bone, wherein the thread comprises a lower flank that faces the tip and an upper flank that faces away from the tip, and wherein the upper flank and the lower flank form a second angle with each other; and
removing material from both the upper flank and the lower flank along a portion of the thread to form a first section where the upper flank and the lower flank form a first angle with each other that is smaller than the second angle, while at least part of a remaining portion of the thread where material is not removed defines a second section where the upper flank and the lower flank continue to form the second angle with each other.

19. The method of claim 18, wherein the first section of the thread is closer to the tip than the second section of the thread is to the tip.

20. A bone screw comprising:
a shank configured to be anchored in bone, the shank having a longitudinal axis and comprising a tip configured to be inserted first into bone, a core, and a thread with a plurality of turns winding in a helix around at least a portion of the core to engage bone;
a threadless neck at an end of the shank opposite the tip; and
a head connected to the shank by the neck and having a width measured perpendicular to the longitudinal axis that is greater than a maximum width of the neck;
wherein the thread comprises a lower flank that faces the tip and an upper flank that faces away from the tip, wherein for a first section of the thread, the upper flank and the lower flank form a first angle with each other, and wherein for a second section of the thread directly connected to the first section and located between the first section and the neck, respective angles formed between each of the upper flank and the lower flank with the longitudinal axis are different from those of the first section such that the upper and lower flanks form a second angle with each other that is greater than the first angle.

21. The bone screw of claim 20, wherein an outer diameter of the second section of the thread adjacent to the first section of the thread is at least as wide as an outer diameter of the first section of the thread adjacent to the second section of the thread.

22. The bone screw of claim 20, wherein the first and second sections of the thread include a same number of helices as one another.
